(19) United States
(12) Patent Application Publication
(10) Pub. No.: US 2023/0029984 A1
(43) Pub. Date: Feb. 2, 2023

(54) CORONARY COVERED STENT

(71) Applicant: Merit Medical Systems, Inc., South Jordan, UT (US)

(72) Inventors: Christopher Cindrich, Highland, UT (US); John Hall, Bountiful, UT (US); Wayne Mower, Bountiful, UT (US)

(21) Appl. No.: 17/816,301

(22) Filed: Jul. 29, 2022

Related U.S. Application Data
(60) Provisional application No. 63/228,438, filed on Aug. 2, 2021.

(51) Int. Cl. A61F 2/07

(52) U.S. Cl. CPC A61F 2/07; A61F 2002/91575

(57) ABSTRACT

A medical appliance or prosthesis comprising a support structure and a covering is disclosed. The support structure includes a frame comprising a wire winding. The wire winding includes an end row including apexes and troughs. The apexes and troughs are configured to allow crimping of the frame to a low profile while reducing strain on the wire. The covering includes a multilayer construct including fibrous inner and outer layers and a barrier layer.

### RELATED APPLICATIONS

This application claims priority to U.S. Provisional Application No. 63/228,438, filed on Aug. 2, 2021 and titled, “Coronary Covered Stent,” which is hereby incorporated by reference in its entirety.

### TECHNICAL FIELD

The present disclosure relates generally to medical devices. More specifically, the present disclosure relates to covered medical prostheses. In some embodiments, the present disclosure relates to vascular covered stents, including coronary covered stents.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of an embodiment of a vascular prosthesis.

FIG. 1B is a cross-sectional view of the vascular prosthesis of FIG. 1A taken through line 1B-1B.

FIG. 2 is a front view of an embodiment of a support structure of the vascular prosthesis of FIG. 1A.

FIG. 3A is a lay flat view of another embodiment of a support structure of another embodiment of a vascular prosthesis in an expanded state.

FIG. 3B is a lay flat view of the support structure of FIG. 3A in a crimped state.

FIG. 4A is a lay flat view of another embodiment of a support structure of another embodiment of a vascular prosthesis in an expanded state.

FIG. 4B is a lay flat view of the support structure of FIG. 4A in a crimped state.

FIG. 5A is a lay flat view of another embodiment of a support structure of another embodiment of a vascular prosthesis in an expanded state.

FIG. 5B is a lay flat view of the support structure of FIG. 5A in a crimped state.

### DETAILED DESCRIPTION

Medical prostheses, such as stents, may be deployed in various body lumens for a variety of purposes. Stents may be deployed, for example, in the vascular system for a variety of therapeutic purposes including the treatment of occlusions or stenoses within the lumens of that system. The current disclosure may be applicable to covered stents designed for treatment of the central venous (“CV”) system, the peripheral vascular (“PV”) system, abdominal aorta, bronchus, esophagus, the biliary system, the coronary system, the gastrointestinal system, the neuro vascular system, thoracic aorta, or any other system with a lumen.

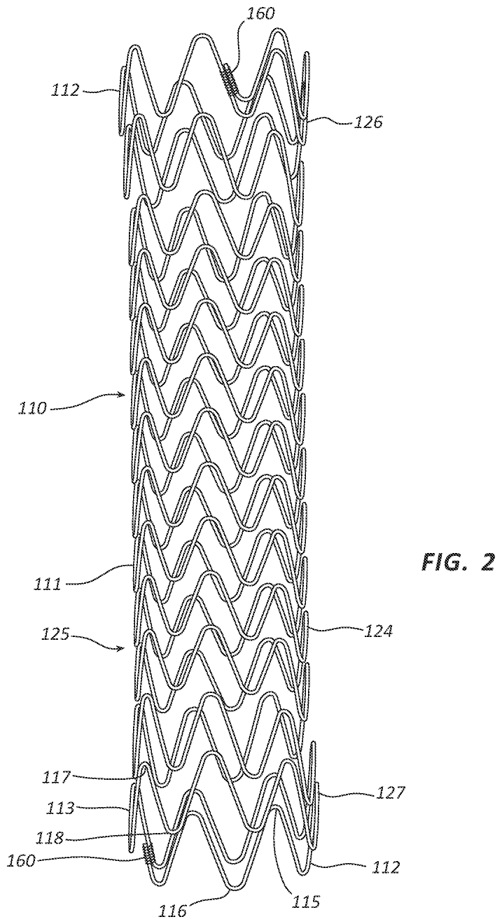

The current disclosure relates to medical prostheses, including covered stents, which may comprise a support structure provided in connection with one or more coverings or coatings. Though particular structures and coverings are described herein, any feature of the structures or coverings described herein may be combined with any other disclosed feature without departing from the scope of the current disclosure. For example, certain figures referenced below show a frame of a support structure without any covering; the features described and illustrated in those figures may be combined with any covering disclosed herein. Further, as used herein, the term “frame” refers to a support structure for use in connection with a prosthesis. For instance, a support structure, such as that described in connection with FIG. 2, below, is an example of a frame used in connection with a medical prosthesis. In some embodiments, a medical prosthesis—such as a stent—may comprise the frame alone, with no covering or other components.

As used herein, the term “stent” refers to a medical prosthesis configured for use within a bodily structure, such as within a body lumen. A stent may comprise a scaffolding or support structure, such as a frame, and/or a covering. Thus, as used herein, “stent” refers to both covered and uncovered support structures.

The components of the embodiments as generally described and illustrated in the figures herein could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a stent or another medical appliance. The proximal end of a prosthesis is defined as the end closest to the practitioner when the prosthesis is disposed within a deployment device which is being used by the practitioner. The distal end is the end opposite the proximal end, along the longitudinal direction of the appliance, or the end furthest from the practitioner. It is understood that, as used in the art, these terms may have different meanings once the appliance is deployed (i.e., the “proximal” end may refer to the end closest to the head or heart of the patient depending on application). For consistency, as used herein, the ends labeled “proximal” and “distal” prior to deployment remain the same regardless of whether the appliance is deployed. The longitudinal direction of a stent is the direction along the axis of a generally tubular stent. In embodiments where a stent or another appliance is composed of a wire structure coupled to one or more layers of a film or sheet-like components, such as a polymer layer, the metal structure is referred to as the “support structure” or “frame,” and the polymer layer as the “covering” or “coating.”

The term “covering” may refer to a single layer of polymer, multiple layers of the same polymer, or layers comprising distinct polymers used in combination. Furthermore, as used herein, the term “covering” refers only to a layer or layers which are coupled to a portion of the support structure; neither term requires that the entire support structure be “covered.” In other words, medical appliances wherein a portion of the support structure may be covered and a portion remains bare, are within the scope of this disclosure.

Medical device coverings may comprise multilayered constructs, comprised of two or more layers which may be serially applied. Further, multilayered constructs may comprise nonhomogeneous layers, meaning adjacent layers have differing properties. Thus, as used herein, each layer of a multilayered construct may comprise a distinct layer, either due to the distinct application of the layers or due to differing properties between layers.

Additionally, as used herein, “tissue ingrowth” or “cellular penetration” refer to any presence or penetration of a biological or bodily material into a component of a medical prosthesis. For example, the presence of body tissues (e.g., collagen, cells, and so on) within an opening or pore of a layer or component of a medical prosthesis comprises tissue ingrowth into that component. Further, as used herein, “attachment” of tissue to a component of a medical prosthesis refers to any bonding or adherence of a tissue to the appliance, including indirect bonds. For example, tissue of some kind (e.g., collagen) may become attached to a stent covering (including attachment via tissue ingrowth) and another layer of biological material (such as endothelial cells) may, in row, adhere to the first tissue. In such instances, the second biological material (endothelial cells in the example) and the tissue (collagen in the example) are “attached” to the stent covering.

Furthermore, through the present disclosure, certain fibrous or porous materials may be referred to as inhibiting or promoting certain biological responses. These relative terms are intended to reference the characteristics of the fibrous materials with respect to non-fibrous materials or coatings. Examples of non-fibrous coatings include non-fibrous PTFE sheets, other similarly formed polymers, and the like. Examples of fibrous coatings include rotational spun PTFE, electrospun PTFE, expanded PTFE, and other similarly formed polymers or materials. Examples of spun fibrous coatings include rotational spun PTFE, electrospun PTFE, and other similarly formed polymers or materials, and exclude expanded PTFE.

Lumens within the vascular system are generally lined with a single layer (monolayer) of endothelial cells. This lining of endothelial cells makes up the endothelium. The endothelium acts as an interface between blood flowing through the lumens of the vascular system and the inner walls of the lumens. The endothelium, among other functions, reduces or prevents turbulent blood flow within the lumen. The endothelium plays a role in many aspects of vascular biology, including atherosclerosis, creating a selective barrier around the lumen, blood clotting, inflammation, angiogenesis, vasoconstriction, and vasodilation.

A therapeutic medical prosthesis which includes a covering of porous or semi-porous material may permit the formation of an endothelial layer onto the porous surface of the blood contact side of the medical device. Formation of an endothelial layer on a surface, or endothelialization, may increase the biocompatibility of an implanted device. For example, a stent which permits the formation of the endothelium on the inside diameter (blood-contacting surface) of the stent may further promote healing at the therapeutic region and/or have longer-term viability. For example, a stent coated with endothelial cells may be more consistent with the surrounding body lumens, thereby resulting in less turbulent blood flow or a decreased risk of thrombosis, or the formation of blood clots. A stent which permits the formation of an endothelial layer on the inside surface of the stent may therefore be particularly biocompatible, resulting in less trauma at the point of application, fewer side effects, and/or longer-term device viability. Medical prostheses including a covering of porous or semi-porous material may be configured to inhibit or reduce inflammatory responses by the body toward the tissue-contacting side of the medical appliance, for example. Mechanisms such as an inflammatory response by the body toward the medical appliance may stimulate, aggravate, or encourage negative outcomes, such as neointimal hyperplasia. For example, a stent configured to permit tissue ingrowth and/or the growth or attachment of endothelial cells onto the blood-contacting side of the device may reduce the likelihood of negative flow characteristics and blood clotting. Similarly, a stent so configured may mitigate the body's inflammatory response toward the material on, for example, the tissue or non-blood-contacting side of the device. By modulating the evoked inflammatory response, negative outcomes such as the presence of bioactive inflammatory macrophages and foreign body giant cells may be reduced. This may aid in minimizing the chemical chain of responses that may encourage fibrous capsule formation surrounding the device and events stimulating neointimal hyperplasia.

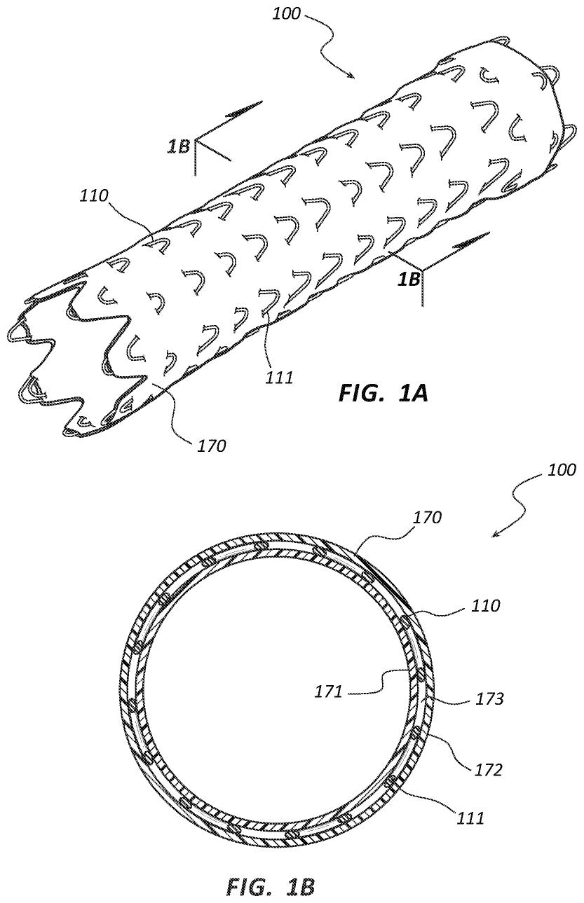

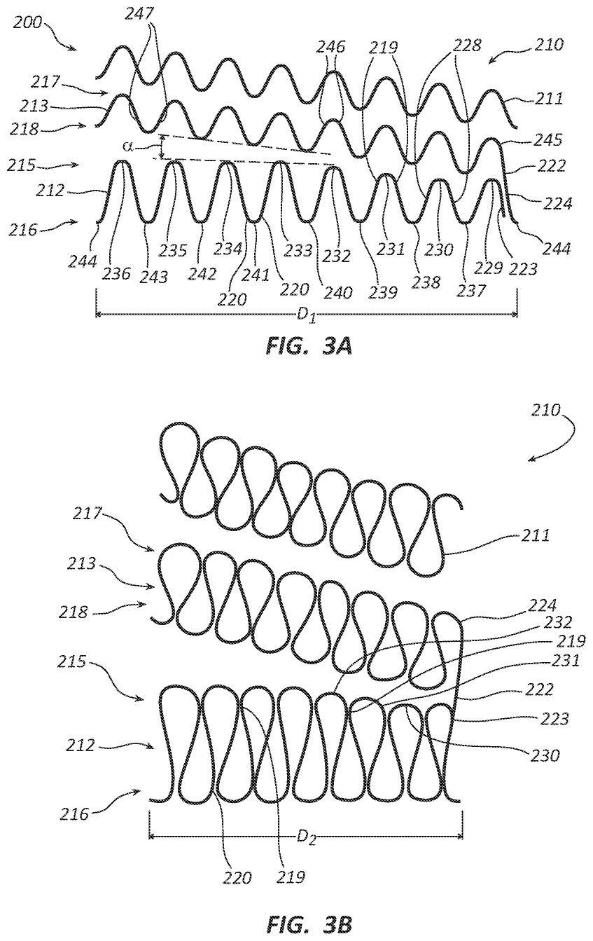

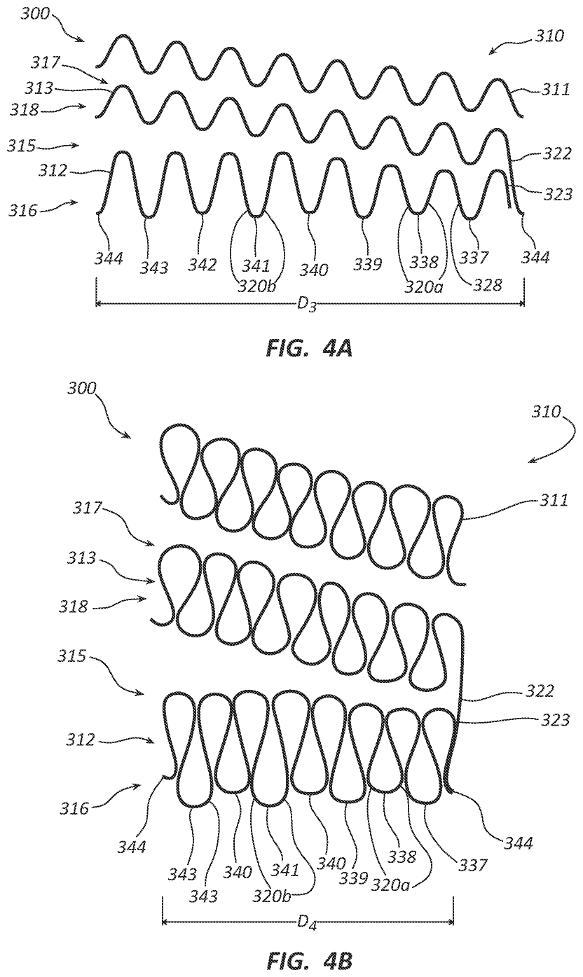

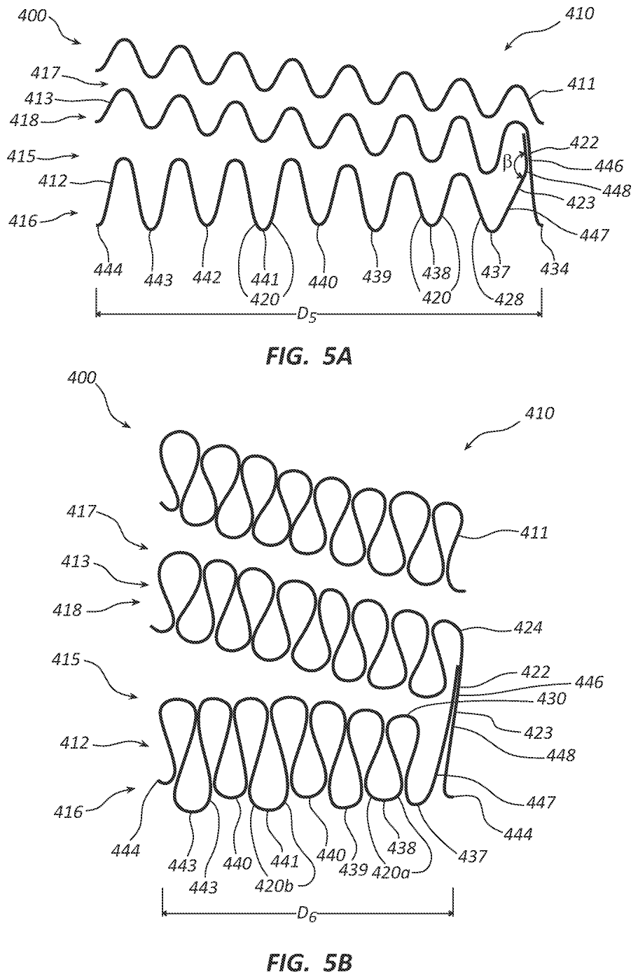

FIGS. 1A and 1B illustrate an embodiment of a vascular prosthesis having a support structure. FIG. 2 illustrates an embodiment of a frame of the support structure of FIG. 1A. FIGS. 3A and 3B illustrate another embodiment of a frame of another embodiment of a vascular prosthesis. FIGS. 4A and 4B illustrate another embodiment of a frame of another embodiment of a vascular prosthesis. FIGS. 5A and 5B illustrate another embodiment of a frame of another embodiment of a vascular prosthesis. Regardless of whether a vascular prosthesis illustrated in any particular figure is illustrated with a particular frame, covering, or without any covering or at all, any embodiment of the vascular prosthesis may be configured with any of the combinations of frames or coverings shown or described herein.

FIG. 1A illustrates an embodiment of a vascular prosthesis 100. In some embodiments, the vascular prosthesis 100 may be configured for use as a vascular stent graft. In other embodiments, the vascular prosthesis 100 can be configured for use as a coronary stent graft. In the illustrated embodiment, the vascular prosthesis 100 comprises a support structure or stent 110 including a frame 111 and a covering 170.

FIG. 1B illustrates a cross-sectional view of the vascular prosthesis 100, again comprising the support structure 110 including the frame 111 and the covering 170. The covering 170 is shown to comprise an inner layer 171, an outer layer 172, and a middle or tie layer 173. Though in the illustration of FIG. 1B the tie layer 173 is shown at the same “level” as the support structure 110, the tie layer 173 may be above or below the support structure 110 in some embodiments. Further, as shown in FIG. 1B, each layer of the covering 170 may be disposed so that there are no voids between layers. In certain embodiments, the covering 170 may have more or fewer layers than the illustrated embodiment, including embodiments with only one covering layer.

FIG. 2 illustrates a front elevation view of an embodiment of the frame 111 of the support structure 110. The illustrated embodiment depicts one embodiment of a configuration for a wire 124 forming the frame 111. As depicted in FIG. 2, the frame 111 may consist of a single continuous wire. In some embodiments, frames, analogous to frame 111, for use in connection with the vascular prosthesis 100 may be fabricated or formed into particular geometries through a variety of techniques. For example, a frame may be cut from a single tube of material, including embodiments wherein the frame is first laser cut, then expanded. In other embodiments, a frame may be molded, including embodiments wherein the frame is molded from a polymeric material. In still other embodiments, powder metallurgical processes, such as powdered compression molding or direct metal laser sintering, may be used.

Referring to FIG. 2, the illustrated frame 111 comprises the wire 124 shaped to form the frame 111. The wire 124 may be shaped in a helical wave-type configuration. Other suitable frame configurations are within the scope of this disclosure. The frame 111 can include an end row 112 disposed at both proximal and distal ends 126, 127 of the frame 111. In some embodiments, the end row 112 has a similar geometry at both ends. In other embodiments, the end row 112 includes a first geometry at one end and a second geometry at the opposing end of the frame 111. The waves of the end row 112 define apexes 115 and troughs 116. In the depicted embodiment, the number of apexes 115 is eight and the number of troughs 116 is eight. In other embodiments, the number of apexes 115 and troughs 116 can range from four to twelve. In some embodiments, the number of apexes 115 may not be equivalent to the number of troughs 116.

The frame 111 can also include at least one body row 113 disposed between the end rows 112. In certain embodiments, the frame 111 includes a plurality of body rows 113 disposed between the end rows 112. The waves of the body rows 113 define apexes 117 and troughs 118. In the depicted embodiment, the apexes 117 are axially aligned with the apexes 115 and the troughs 118 are axially aligned with the troughs 116. In other embodiments, the apexes 117 and troughs 118 may be circumferentially offset from the apexes 115 and the troughs 116, respectfully. In the depicted embodiment, the number of apexes 117 is eight and the number of troughs 118 is eight. In other embodiments, the number of apexes 117 and troughs 118 can each range from four to twelve. In some embodiments, the number of apexes 115 and troughs 116 of the end row 112 may or may not be equivalent to the number apexes 117 and troughs 118 of the body row 113.

The frame 111 may be designed such that a midsection 125 is “harder” than proximal and distal end sections 126, 127. The “hardness” of the frame 111 refers to the relative strength of the structure (e.g., its compressibility). A harder portion of the frame 111 will have greater strength (i.e., exert a greater radial outward force) than a softer portion. In one embodiment, the midsection 125 is harder than the proximal and distal end sections 126, 127 which are relatively softer to prevent trauma to the vessel wall. Further, the frame 111 may be configured to be flexible to facilitate the ability of the vascular prosthesis 100 to conform to the native anatomy at which the vascular prosthesis 100 is configured for use.

The frame 111 may be formed in a variety of sizes. In some embodiments, a length L may range from about 5 millimeters to about 50 millimeters. For example, in coronary applications the length L may range from about 5 millimeters to about 25 millimeters or any value between. In PV applications the length L may range from about 10 millimeters to about 250 millimeters. The frame 111 may also be longer or shorter than these exemplary values in other applications.

The frame 111 may be formed from the single continuous wire 124. In some embodiments the wire 124 may be comprised of Nitinol (ASTM F2063), or other suitable materials. In some embodiments the wire 124 has a diameter between about 0.025 millimeter and about 1.27 millimeter, including from about 0.127 millimeter and about 0.635 millimeter. For example, in some frames designed for CV or PV application, the wire diameter may be from about 0.203 millimeter to about 0.305 millimeter including certain embodiments where the wire is from about 0.229 millimeter to about 0.279 millimeter in diameter or embodiments where the wire is about 0.254 millimeter in diameter. Furthermore, frames configured for the coronary arteries may be formed of wires up to 0.152 millimeter in diameter, including wires between about 0.076 millimeter and 0.127 millimeter in diameter.

Furthermore, in some embodiments the frame 111 may be configured with radiopaque markers 160 at one or more points along the frame 111. Such markers 160 may be crimped or otherwise attached to the frame 111. In other embodiments a radiopaque ribbon, for example a gold ribbon, may be threaded or applied to the frame 111. In certain embodiments, the radiopaque ribbon can be positioned under or over the frame 111 by attachment to the covering 170. In some embodiments the radiopaque markers 160 may be located at or adjacent to one or both the proximal and distal ends 126, 127 of the frame 111. Any radiopaque material may be used, for example gold or tantalum. The radiopaque markers 160 may be configured to facilitate the delivery and placement of the vascular prosthesis 100 and/or to facilitate viewing of the vascular prosthesis 100 under fluoroscopy or X-ray imaging.

In certain instances, a frame may be configured to allow a vascular prosthesis to be crimped into a relatively low-profile configuration for delivery to a vascular treatment site with acceptable strain on a wire of the frame to prevent failure or breakage of the frame in vivo. Crimping is defined herein as radially compressing an expanded or non-constrained vascular prosthesis to reduce a diameter of the vascular prosthesis. For example, medical devices of a certain diameter or constrained low-profile are more feasible for delivery at certain vascular or other access points than others. For example, in many instances a device configured for insertion via the radial artery having a lumen diameter of about two millimeter to three millimeter and delivered to a coronary artery having a lumen diameter of about two millimeters to about four millimeters may be crimped to a relatively smaller diameter than devices configured for insertion via the generally larger femoral artery having a lumen diameter of about eight millimeters to about 10 millimeters and delivered to a popliteal artery having a lumen diameter of about six millimeters to about eight millimeters.

The vascular prosthesis 100 may be configured to be crimped to a particular diameter or low-profile to enable potential access at various or desired access points and delivery to various and desired delivery points via a delivery catheter ranging in size from about 3 French to about 24 French, including from about 3 French to about 16 French and from about 3 French to about 16 French, where one French is equivalent to one third of a millimeter. In some embodiments, the vascular prosthesis 100 may be crimped to a diameter ranging from about one millimeter to about eight millimeters to be co-axially disposed within the delivery catheter. For example, the vascular prosthesis 100 may have an expanded diameter of about four millimeters to about five millimeters and be crimped to a diameter of about 1.3 millimeters to about 1.7 millimeters to fit within a 4 French to 5 French delivery catheter. Once the vascular prosthesis 100 is positioned within the body it may be expanded or deployed in a number of ways, including use of self-expanding materials and configurations. Additionally, some configurations may be designed for expansion by a secondary device, such as a balloon.

Once the vascular prosthesis 100 is expanded or deployed, for example within a coronary artery, the wire 124 of the frame 111 may be exposed to repeated flexing and/or twisting as the heart beats. The repeated flexing and/or twisting can cause metal fatigue or other modes of failure or breakage of the wire 124 and the frame 111 leading to failure of the vascular prosthesis 100 and restenosis of the coronary artery. The breakage of the wire 124 may occur following fewer heart beats if the wire 124 is unacceptably strained when the vascular prosthesis 100 is crimped for delivery.

In some embodiments, the crimp diameter to which the vascular prosthesis 100 can be reduced may be achieved by controlling certain design parameters of the frame 111 to achieve a high packing density and/or high percent diameter reduction with a low strain on the wire 124. Packing density is defined herein as a wire cross-sectional area of a frame per diameter of the frame in the crimped state. For example, the packing density of a frame having a large wire cross-sectional area and a small crimped diameter is larger than the packing density of a frame with the same wire cross-sectional area but larger crimped diameter. The design parameters that can impact the crimped diameter and packing density include the positioning of apexes and troughs of a frame, as is described below.

FIGS. 3A and 3B depict an embodiment of a vascular prosthesis 200 that resembles the vascular prosthesis 100 described above in certain respects. Accordingly, like features are designated with like reference numerals, with the leading digit incremented to “2.” For example, the embodiment depicted in FIGS. 3A and 3B includes a frame 211 that may, in some respects, resemble the frame 111 of FIG. 2. Relevant disclosure set forth above regarding similarly identified features thus may not be repeated hereafter. Moreover, specific features of the vascular prosthesis 100 and related components shown in FIGS. 1A-2 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the vascular prosthesis 200 and related components depicted in FIGS. 3A and 3B. Any suitable combination of the features, and variations of the same, described with respect to the vascular prosthesis 100 and related components illustrated in FIGS. 1A-2 can be employed with the vascular prosthesis 200 and related components of FIGS. 3A and 3B, and vice versa. This pattern of disclosure applies equally to further embodiments depicted in subsequent figures and described hereafter, wherein the leading digits may be further incremented

FIG. 3A illustrates another embodiment of a vascular prosthesis 200 including a frame 211 of a support structure 210 in a lay flat form. As illustrated, the frame 211 includes an end row 212 comprising a plurality of apexes 215 comprising apexes 229, 230, 231, 232, 233, 234, 235, and 236 and a plurality of troughs 216 comprising troughs 237, 238, 239, 240, 241, 242, 243, and 244 that define an expanded diameter D₁. Each of the apexes 215 includes a radius that is substantially equivalent to a radius of each of the troughs 216. A strut 228 is disposed between an adjacent apex and trough. For example, struts 228 are disposed between trough 237 and apex 230 and between apex 230 and trough 238. Each of the apexes 215 include an apex shoulder 219 disposed on opposing sides of the apexes 215. Each of the troughs 216 include a trough shoulder 220 disposed on opposing sides of the troughs 216. The troughs 216 are circumferentially aligned such that the trough shoulders 220 are also circumferentially aligned.

The apexes 229, 230 have substantially equivalent heights or amplitudes relative to the trough 237. The apex 231 has a greater height than the apex 230 relative to the trough 238 and the apex 232 has a greater height than the apex 231 relative to the trough 239 such that the apex shoulders 219 of apex 231 and apex 232 are not circumferentially aligned but are axially offset. The apexes 233, 234, 235, and 236 have substantially equivalent heights relative to the troughs 240, 241, 242, and 243, respectively, such that their apex shoulders 219 are circumferentially aligned. An end strut 223 of the end row 212 extends from the apex 229 away from a body row 213 of the frame 211 and is not coupled to a trough.

A transition strut 222 is disposed between the trough 244 and an apex 245 of the body row 213 and is oriented substantially parallel to the end strut 223. The transition strut 222 can be configured to transition a wire 224 from the end row 212 to the body row 213 over a short circumferential distance. The short circumferential distance can include anything from a single apex 245 to all apices 245 of a full revolution around the circumference of the frame 211. In some embodiments, the short circumferential distance may include more than a partial or full revolution around the circumference of the frame 211. Said another way, the transition strut 222 can axially transition the body row 213 away from end row 212 over an arc extending along a portion of the circumference of the frame 211. The transition strut 222 can axially offset the body row 213 from the end row 212 such that a plurality of troughs 218 of the body row 213 are not nested within the troughs 216 of the end row 212 and a plurality of apexes 215 of the end row 212 are not nested within the apexes 217 of the body row 213.

The transition strut 222 can be attached to the end strut 223 using any suitable technique. For example, in one embodiment, the transition strut 222 can be attached to the end strut 223 using a string or wire, such as a radiopaque wire, to tie or bind the transition strut 222 and the end strut 223 together. The radiopaque wire can be used to identify a location of the end of the vascular prosthesis 200 when positioning the vascular prosthesis 200 within a patient using an X-ray or fluoroscopy imaging technique. In other embodiments, the transition strut 222 may be coupled to the end strut 223 using any other known suitable technique, such as laser welding, mechanical crimping, etc. In embodiments where the frame 211 is an element of the vascular prosthesis 200 further comprising a covering, the end strut 223 may be secured relative to the transition strut by being coupled or bound to the covering.

The body row 213 comprises the apexes 217 and the troughs 218. The apexes 217 include apex shoulders 246 and the troughs 218 include trough shoulders 247. A pitch angle α of the body row 213 relative to a transverse axis of the frame 211, may allow the apex shoulders 246 to be axially offset from adjacent apex shoulders 246 and the trough shoulders 247 to be axially offset from adjacent trough shoulders 247.

FIG. 3B is a lay flat illustration of the frame 211 of the support structure 210 in a crimped state. As shown, the trough shoulders 220 of the troughs 216 are circumferentially aligned and brought into contact with one another to define a crimp diameter D₂. The apex shoulders 219 of apexes 230, 231, and 232 are axially offset and not circumferentially aligned with adjacent apex shoulders 219 when the frame 211 is in the crimped state. Further, the transition strut 222 is shown to axially offset the troughs 218 of the body row 213 away from the apexes 215 of the end row 212 such that the troughs 218 are not nested within the troughs 216. Disposing of the troughs 218 away from the troughs 216 and axially offsetting apex shoulders 219 can decrease a wire cross-sectional area of a transverse cross-section and packing density of the end row 212 and allow the end row 212 to be crimped to the diameter D₂ with reduced strain applied to the wire 224 adjacent the troughs 216 when compared to a frame having troughs of a body row nested within troughs of an end row and troughs of the end row circumferentially aligned. The crimp diameter D₂ can be from about 20% to about 100% of the expanded diameter D₁.

FIG. 4A is a lay flat illustration of another embodiment of a vascular prosthesis 300 including a frame 311 of a support structure 310 in an expanded state. As illustrated, the frame 311 includes a plurality of troughs 316 of an end row 312 that are not circumferentially aligned that define an expanded diameter D3. The troughs 316 include troughs 337, 338, 339, 340, 341, 342, 343, and 344. The troughs 316 are alternatingly long and short relative to respective apexes 315. For example, troughs 337, 339, 341, and 343 are long and troughs 338, 340, 342, and 344 are short. In other words, the short troughs 338, 340, 342, and 344 are axially offset towards a body row 313 relative to the troughs 337, 339, 341, and 343. A length of the axial offset may range from about 1.0 times to about 1.5 times a radius of the troughs 316. The struts 328 of the long troughs 337, 339, 341, and 343 are longer than the struts of the short troughs 338, 340, 342, and 344. Trough shoulders 320a of the short troughs 338, 340, 342, and 344 are axially offset towards the body row 313 relative to trough shoulders 320b of the long troughs 337, 339, 341, and 343. In another embodiment, the apexes 315 may be configured in a similar manner as the troughs 316. For example, a first apex of the apexes 315 may be long and an adjacent apex may be short relative to a respective trough.

FIG. 4B is a lay flat illustration of the frame 311 in a crimped state. As illustrated, the trough shoulders 320 of the short troughs 338, 340, 342, and 344 are axially offset from and do not contact the trough shoulders 320 of the long troughs 337, 339, 341, and 343. Offsetting the shoulders 320 of the short troughs 338, 340, 342, and 344 relative to the shoulders 320 of the long troughs 337, 339, and 341, can decrease a wire cross-sectional area of a transverse cross-section of the end row 312 and packing density to allow the end row 312 to be crimped to the diameter D₄ with reduced strain applied to the wire 324 adjacent the troughs 316 when compared to a frame having shoulders of troughs of an end row circumferentially aligned.

FIG. 5A is a lay flat illustration of another embodiment of a vascular prosthesis 400 including a frame 411 in an expanded state. As illustrated the frame 411 is expanded to a diameter D₅. The frame 411 comprises an end strut 423 of an end row 412 that extends from a trough 437 toward a body row 413. The trough 437 can have a radius that is larger than a radius of an adjacent trough. The frame 411 can comprise a plurality of apexes 415 that number one less than a number of a plurality of troughs 416. For example, in the illustrated embodiment, the number of apexes 415 is seven and the number of troughs 416 is eight. The end strut 423 includes a free-end section 446, a trough section 447, and a bend 448 disposed between the sections 446, 447. The bend 448 may include an obtuse angle β ranging from greater than 90 degrees to less than 180 degrees. A transition strut 422 can be coupled to the free-end section 447 using any suitable technique, such as any of the techniques previously described. The end strut 423 may provide a softer, more compressible end portion of the frame 411 because the trough section 447 is not supported by the transition strut 422 and the obtuse angle β is weak allowing for flexibility of the trough section 447.

FIG. 5B is a lay flat illustration of the frame 411 in a crimped state. As illustrated, the end row 412 is crimped to a diameter D₆. The end strut 423 extends from the trough 437 toward the body row 413 and is displaced toward an apex 430. The number of the apexes 415 is one less than the number of the troughs 416. The reduction of the number of apexes 415 by one relative to the number of troughs 416 of the end row 412 can decrease a wire cross-sectional area of a transverse cross-section of the end row 412 and packing density to allow the end row 412 to be crimped to the diameter D₆ with reduced strain applied to the wire 424 adjacent the apexes 415 when compared to a frame having an equal number of apexes and troughs of an end row. The crimp diameter D₆ can be from about 20% to about 100% of the expanded diameter D₅.

Referring again to FIGS. 1A and 1B, the covering 170, as previously stated, comprises the first or inner layer 171, the second or outer layer 172, and the tie layer 173. As depicted in FIG. 1B, the inner layer 171 defines a luminal surface of the vascular prosthesis 100, the outer layer 172 defines an abluminal surface, and the tie layer 173 and the support structure 110 are disposed between the inner layer 171 and the outer layer 172.

In certain embodiments, a relatively porous inner layer 171 and outer layer 172 may be desirable. The relatively porous inner and outer layers 171, 172 may permit tissue ingrowth and/or endothelial attachment or growth on the luminal surface and abluminal surface of the vascular prosthesis 100 which may be desirable for any combination of the following: healing, anchoring, biocompatibility, prevention of thrombosis, and/or reducing turbulent blood flow within the vascular prosthesis 100.

In certain embodiments, the inner layer 171 can be porous and permeable to ingrowth or migration of cells (e.g., endothelial cells) into or through the inner layer 171 to form a luminal surface that is resistant to thrombus formation within a lumen of the vascular prosthesis 100. When a thrombus forms within the lumen, the lumen may be occluded and blood flow through the vascular prosthesis 100 may be either restricted or prevented. In other embodiments, the inner layer 171 may be impermeable to migration of endothelial cells into or through the inner layer 171.

In some embodiments, the inner layer 171 can include serially deposited micro or nano fibers produced through a rotational spinning process (rspin). For example, a flowable polymer dispersion of solution may be loaded into a cup or spinneret configured with orifices on an outside circumference of the spinneret. The orifices may be 29 gage or 30 gage needles. The solution or dispersion may include from about 5 weight % to about 70 weight % polymer and from about 60 weight % to about 70 weight %; and from about 0.05 weight % to about 15 weight %, from about 1 weight % to about 5 weight %, and from about 0.1 weight % to about 0.2 weight % additive particles. The spinneret is then rotated at a rate of about 5500 rpm to about 6500 rpm for about three minutes, causing (through a combination of centrifugal and hydrostatic forces, for example) the material within the cup or spinneret to be expelled from the orifices. The material may then form a “jet” or “stream” extending from the orifice, with drag forces tending to cause the stream of material to elongate into a small diameter fiber. The fibers may then be directly deposited on a collection apparatus to form a sheet or a covering. In some instances, and with some materials, the sheet may then be sintered, for example at a temperature between about 360° C. and about 400° C. and about 385° C. for about 8 min. In some embodiments, the rotational spinning process is completed in the absence of an electrical field. Exemplary methods and systems for rotational spinning can be found in U.S. Patent Publication No. US2009/0280325, titled “Methods and Apparatuses for Making Superfine Fibers,” which is incorporated herein by reference in its entirety.

In other embodiments, the serially deposited micro or nano fibers of the inner layer 171 may be produced through an electrospinning process (espin). For example, a flowable polymer (e.g., a PTFE dispersion or other polymer solution) may be loaded into a syringe pump or other device configured to expel the materials through an orifice. In some embodiments, the polymer dispersion or solution may include carbon material particles (e.g., graphene) and/or therapeutic micro particles. The solution or dispersion may include from about 5 weight % to about 70 weight % polymer and from about 0.05 weight % to about 1.0 weight % additive particles. The solution is dispensed from the orifice at a controlled rate and electrostatic forces are used to draw the expelled material to a collection apparatus. The electrostatic force can be about 1.5 kV. The material may then form a “jet” or “stream” extending from the orifice. In some instances, the orifice or solution may be charged and an opposite electrostatic charge is applied to the collection surface such that a difference in electrostatic charge causes the stream of material to elongate into a small diameter fiber 120. The fibers 120 may then be directly deposited on the collection apparatus that is about seven inches from the orifice to form the mat 110. For some materials, the mat 110 may then be sintered at a temperature between about 360° C. and about 400° C. and about 385° C. for about 8 minutes. Electrospinning is described in U.S. patent application Ser. No. 13/360,444, titled “Electrospun PTFE Coated Stent and Method of Use,” which is incorporated herein by reference in its entirety.

In some embodiments, a pressure extrusion and stretching process may produce a porous expanded material used in the outer layer 172. The process may comprise the steps of: (a) mixing a polymer (e.g., PTFE) at a concentration of from about 70 weight % to about 95 weight %, a lubricating agent at a concentration of from about 5 weight % to about 30 weight %, such that a lube/polymer ratio ranges from about 5% to about 30%, and the additive particles at a concentration of from about 0.01 weight % to about 5 weight % to form a solution or dispersion; (b) forming a billet comprising the solution or dispersion; (c) extruding the billet under a pressure of about 300 pounds to about 1000 pounds at a rate of about 0.01 inch/minute to about 0.3 inch/minute and at a temperature of about 21° C. to about 70° C. to form a tape; (d) calendaring and/or drying the tape to facilitate evaporation of the lubricating agent; (e) tentering the tape to uniaxially or biaxially stretch it in a first direction at about one inch/sec to about 30 inches/second to about 110% to about 600% elongation and/or a second direction perpendicular to the first direction to form nodes and fibrils, and/or stretching the material in one or more directions through other processes; and (f) sintering the material, for example at a temperature between about 360° C. and about 400° C. In some embodiments, the solution or dispersion may include carbon material particles (e.g., graphene) and/or therapeutic micro particles.

A variety of materials may be either rotational spun, electrospun, or extruded and stretched. For example, these materials include polytetrafluoroethylene (PTFE), fluorinated ethylene propylene (FEP), Dacron or polyethylene terephthalate (PET), polyurethanes, polycarbonate polyurethanes, polypropylene, Pebax, polyethylene, biological polymers (such as collagen, fibrin, and elastin), and ceramics among other materials.

Furthermore, additives or active agents may be integrated with the rotational spun, electrospun, or extruded and stretched materials including instances where the additives are directly rotational spun, electrospun, or extruded and stretched with other materials. Such additives may include carbon materials such as graphene, radiopaque materials such as bismuth oxide, antimicrobial agents such as silver sulfadiazine, antiseptics such as chlorhexidine or silver and anticoagulants such as heparin. Organic additives or components may include fibrin and/or collagen. In some embodiments, a layer of drugs or other additives may be added to the inner layer 171 and/or the outer layer 172 during manufacture.

The impermeability of the vascular prosthesis may be provided by the tie layer 173 disposed between the outer layer 172 and the inner layer 171. For example, a non-porous, impermeable tie layer 173 may be formed of fluoroethylene propylene (FEP) which is applied, for example, as a film or dip coating between permeable inner and outer layers 171, 172. Furthermore, FEP may be rotational spun or electrospun with a small average pore size to create a substantially cell impermeable layer.

The FEP tie layer 173 may be dip coated on the inner layer 171 by adding 20 ml of water to 50 ml of a 55 weight % FEP dispersion to thin the dispersion. The inner layer 171 may then dipped in the solution to coat the inner layer 171. The tie layer 173 may then be cooked, for example, at 325° C. for 15 minutes. Other concentrations of FEP dispersions for dip coatings are also within the scope of this disclosure. Additionally, polymer dispersions may be sprayed or otherwise applied onto the inner layer 171. Such coatings may be heat treated after application.

In some embodiments the tie layer 173 may be configured to promote bonding between the outer layer 172 and the inner layer 171. In other embodiments the tie layer 173 may further be configured to provide certain properties to the vascular prosthesis 100 as a whole, such as stiffness or tensile strength. The tie layer 173 may thus be configured as a reinforcing layer.

Additionally, in embodiments where both the inner layer 171 and the outer layer 172 are porous in nature, the tie layer 173 may be configured to create an impermeable layer between the two porous layers. In such embodiments the vascular prosthesis 100 may permit tissue ingrowth, tissue attachment and/or healing on both the inner and outer layers 171, 172 while still preventing tissue outside of the vascular prosthesis 100 from growing into the lumen and occluding the lumen. Thus, the tie layer 173 may be configured to inhibit tissue ingrowth into the layer or to be impervious to tissue migration into or through the layer or to substantially inhibit tissue migration.

Furthermore, the tie layer 173 may be configured to be impervious or substantially impervious to fluid migration across the tie layer 173. Specifically, constructions comprising one or more porous layers may allow fluid to cross the porous layer. In the case of a medical appliance configured to control blood flow, such as a graft, a porous layer may allow blood to leak across the layer or may allow certain smaller components of the blood to cross the layer while containing larger components, effectively filtering the blood. In some instances, this filtration or ultrafiltration may allow components such as plasma to cross the barrier while containing red blood cells, leading to seroma. Thus, a fluid impermeable tie layer may be configured to contain fluid within a medical device also comprised of porous layers. In some devices, the tie layer 173 may be both fluid impermeable and impervious to tissue ingrowth or may be configured with either of these properties independent of the other. Constructs wherein any layer (other than, or in addition to the tie layer 173) is configured to be fluid impermeable and/or impervious to tissue ingrowth are also within the scope of this disclosure. Thus, disclosure recited herein in connection with fluid impermeable and/or tissue impervious tie layers 173 may be analogously applied to impermeable layers at various locations within a construct.

The tie layer 173 may include any thermoplastic material. For example, the tie layer 173 may include any of the following polymers or any other thermoplastic: dextran, alginates, chitosan, guar gum compounds, starch, polyvinylpyridine compounds, cellulosic compounds, cellulose ether, hydrolyzed polyacrylamides, polyacrylates, polycarboxylates, polyvinyl alcohol, polyethylene oxide, polyethylene glycol, polyethylene imine, polyvinylpyrrolidone, polyacrylic acid, poly(methacrylic acid), poly(itaconic acid), poly(2-hydroxyethyl acrylate), poly(2-(dimethylamino)ethyl methacrylate-co-acrylamide), poly(N-isopropylacrylamide), poly(2-acrylamido-2-methyl-I-propanesulfonic acid), poly (methoxyethylene), poly(vinyl alcohol), poly(vinyl alcohol) 12% acetyl, poly(2,4-dimethyl-6-triazinylethylene), poly(3morpholinylethylene), poly(N—I,2,4-triazolyethylene), poly (vinyl sulfoxide), poly(vinyl amine), poly(N-vinyl pyrrolidone-co-vinyl acetate), poly(g-glutamic acid), poly(Npropanoyliminoethylene), poly(4-amino-sulfo-aniline), poly [N-(p-sulphophenyl)amino-3-hydroxymethyl-1,4phenyleneimino-I,4-phenylene], isopropyl cellulose, hydroxyethyl, hydroxylpropyl cellulose, cellulose acetate, cellulose nitrate, alginic ammonium salts, i-carrageenan, N-[(3-hydroxy-2′,3-dicarboxy)ethyl]chitosan, konjac glocomannan, pullulan, xanthan gum, poly(allyammonium chloride), poly(allyammonium phosphate), poly(diallydimethylammonium chloride), poly(benzyltrimethylammonium chloride), poly(dimethyldodecyl(2-acrylamidoethyly) ammonium bromide), poly(4-N-butylpyridiniumethylene iodine), poly(2-N-methylpridinium methylene iodine), poly(N methylpryidinium-2,5-diylethenylene), polyethylene glycol polymers and copolymers, cellulose ethyl ether, cellulose ethyl hydroxyethyl ether, cellulose methyl hydroxyethyl ether, poly(I-glycerol methacrylate), poly(2-ethyl-2-oxazoline), poly(2-hydroxyethyl methacrylate/methacrylic acid) 90:10, poly(2-hydroxypropyl methacrylate), poly(2-methacryloxyethyltrimethylammonium bromide), poly(2-vinyl1-methylpyridinium bromide), poly(2-vinylpyridine N-oxide), poly(2-vinylpyridine), poly(3-chloro-2-hydroxypropyl 2-methacryloxyethyldimethylammonium chloride), poly(4vinylpyridine N-oxide), poly(4-vinylpyridine), poly (acrylamide/2-methacryloxyethyltrimethylammonium bromide) 80:20, poly(acrylamide/acrylic acid), poly(allylamine hydrochloride), poly(butadiene/maleic acid), poly(diallyldimethylammonium chloride), poly(ethyl acrylate/acrylic acid), poly(ethylene glycol) bis(2-aminoethyl), poly (ethylene glycol) monomethyl ether, poly(ethylene glycol)bisphenol A diglycidyl ether adduct, poly(ethylene oxide-bpropylene oxide), poly(ethylene/acrylic acid) 92:8, poly(Ilysine hydrobromide), poly(I-lysine hydrobromide), poly (maleic acid), poly(n-butyl acrylate/2methacryloxyethyltrimethylammonium bromide), poly(Niso-propylacrylam ide), poly(N-vinylpyrrolidone/2dimethylaminoethyl methacrylate), dimethyl sulfatequaternary, poly(N-vinylpyrrolidone/vinyl acetate), poly(oxyethylene) sorbitan monolaurate (Tween 20®), poly (styrenesulfonic acid), poly(vinyl alcohol), N-methyl-4(4′formylstyryl)pyridinium, methosulfate acetal, poly(vinyl methyl ether), poly(vinylamine) hydrochloride, poly(vinylphosphonic acid), poly(vinylsulfonic acid) sodium salt, and polyaniline.

Further, in certain embodiments the tie layer 173 may include two or more layers. The tie layer 173 may be formed in any manner known in the art and attached to the inner 171 and outer 172 layers in any manner known in the art. For example, the tie layer 173 may comprise a sheet of material which is wrapped around the inner layer 171 or a tube of material which is slipped over the inner layer 171 which is then heat shrunk or otherwise bonded to the inner and outer layers 171, 172.

While specific embodiments of stents and other medical appliances have been illustrated and described, it is to be understood that the disclosure provided is not limited to the precise configuration and components disclosed. Various modifications, changes, and variations apparent to those of skill in the art having the benefit of this disclosure may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not as a limitation of the scope of the present disclosure in any way. It will be apparent to those having skill in the art, and having the benefit of this disclosure, that changes may be made to the details of the above-described embodiments without departing from the underlying principles of the disclosure herein.

1. A vascular prosthesis, comprising:
a support structure comprising a frame, wherein the frame comprises:
an end row comprising a plurality of apexes and a plurality of troughs;
a body row comprising a plurality of troughs; and
a transition strut disposed between the end row and the body row,
wherein the transition strut is configured to position the body row axially away from the end row, and
wherein the end row is axially offset from the body row when the support structure is radially crimped.

2. The vascular prosthesis of claim 1, wherein the transition strut positions the body row axially away from the end row over an arc extending along a portion of a circumference of the frame.

3. The vascular prosthesis of claim 1, wherein the transition strut is coupled to an end strut of the end row.

4. The vascular prosthesis of claim 3, wherein the end strut is directed axially away from the body row.

5. The vascular prosthesis of any one of claim 1, wherein the plurality of troughs of the end row are circumferentially aligned.

6. The vascular prosthesis of claim 1, wherein at least one of the plurality of troughs of the end row is axially offset from an adjacent trough of the plurality of troughs of the end row, and wherein a shoulder of the at least one of the plurality of troughs of the end row is axially offset from a shoulder of the adjacent trough of the plurality of troughs of the end row when the frame is radially compressed.

7. The vascular prosthesis of claim 1, wherein a height of at least one apex of the plurality of apexes of the end row is shorter than a height of an adjacent apex of the plurality of apexes of the end row.

8. The vascular prosthesis of claim 1, wherein the plurality of apexes of the end row are axially offset from the plurality of troughs of the body row.

9. The vascular prosthesis of claim 1, wherein the support structure further comprises an expanded diameter and a radial compressed diameter, and wherein the radial compressed diameter ranges from 20% to 100% of the expanded diameter.

10. The vascular prosthesis of claim 1, further comprising a cover coupled to the support structure; wherein the cover comprises:
a luminal layer of rotational spun polytetrafluoroethylene (PTFE) fibers;
an abluminal layer of PTFE; and
a tie layer disposed between the luminal and abluminal layers,
wherein the support structure is at least partially disposed between the luminal and abluminal layers and coupled to the tie layer.

11. A vascular prosthesis, comprising:
a support structure comprising a first end portion of a coil comprising:
an end row comprising a plurality of apexes and a plurality of troughs;
a body row comprising a plurality of troughs; and
a transition strut disposed between the end row and the body row,
wherein the transition strut is configured to position the body row axially away from the end row, and
wherein the end row is axially offset from the body row when the support structure is radially compressed; and
a cover coupled to the support structure.

12. The vascular prosthesis of claim 11, wherein the transition strut positions the body row axially away from the end row over an arc extending along a portion of a circumference of the frame.

13. A frame of a vascular support structure, comprising:
an end row comprising a plurality of apexes and a plurality of troughs;
a body row comprising a plurality of troughs; and
a transition strut disposed between the end row and the body row,
wherein the transition strut is configured to position the body row axially away from the end row, and
wherein the end row is axially offset the body row when the support structure is radially compressed.

14. The frame of claim 13, wherein the transition strut positions the body row axially away from the end row over an arc radius extending along a portion of a circumference of the frame.

15. The frame of claim 13, wherein the transition strut is coupled to an end strut of the end row.

16. The frame of claim 13, wherein the frame further comprises a radiopaque marker configured to couple the transition strut to the end strut of the end row.

17. The frame of claim 13, wherein the plurality of troughs of the end row are circumferentially aligned.

18. The frame of claim 13, wherein at least one of the plurality of troughs of the end row is axially offset from an adjacent trough of the plurality of troughs of the end row, and wherein a shoulder of the at least one of the plurality of troughs of the end row is free from contact with a shoulder of the adjacent trough of the plurality of troughs of the end row when the frame is radially compressed.

19. The frame of claim 13, wherein the frame further comprises an expanded diameter and a radial compressed diameter, and wherein the radial compressed diameter ranges from 20% to 100% of the expanded diameter.

20. The frame of claim 13, wherein the frame further comprises any one of a coiled wire and a laser cut tube.
